(19) United States
(12) Patent Application Publication
(10) Pub. No.: US 2022/0023265 A1
(43) Pub. Date: Jan. 27, 2022

(54) USE OF INHIBITORS OF PHOSPHATASE ACTIVITY OF SOLUBLE EPOXIDE FOR THE TREATMENT OF CARDIOMETABOLIC DISEASES

(71) Applicants: INSERM (INSTITUT NATIONAL DE LA SANTÉ ET DE LA RECHERCHE MÉDICALE), Paris (FR); UNIVERSITÉ DE ROUEN NORMANDIE, Mont-Saint-Aignan (FR); CENTRE HOSPITALIER UNIVERSITAIRE DE ROUEN, Rouen (FR); UNIVERSITÉ DE REIMS CHAMPAGNE-ARDENNE, Reims (FR); CENTRE HOSPITALIER UNIVERSITAIRE DE REIMS, Reims (FR)

(72) Inventors: Jérémy BELLIEN, Rouen cedex (FR); Zoubir DJERADA, Reims (FR)

(21) Appl. No.: 17/276,875

(22) PCT Filed: Sep. 16, 2019
(86) PCT No.: PCT/EP2019/074728

§ 371 Date: Mar. 17, 2021

(30) Foreign Application Priority Data
Sep. 17, 2018 (EP) 18306207.4

(51) Int. Cl. A61K 31/421; A61K 31/255; A61K 31/198; A61K 31/407; A61K 31/41; A61P 3/10; A61P 9/10

(52) U.S. Cl. CPC A61K 31/421; A61K 31/255; A61K 31/198; A61P 9/10; A61K 31/41; A61P 3/10; A61K 31/407

(57) ABSTRACT

The growing prevalence of obesity and type 2 diabetes complicates risk and clinical management by potentiating and/or exacerbating hypertension, hyperlipidemia, atherosclerosis and cardiomyopathy, leading to increasing use of the term “cardiometabolic disease” (CMD) to encompass the many facets of this complex syndrome. The inventors assessed the role of the soluble epoxide hydrolase (she) phosphatase domain in metabolism and cardiovascular system, by generating sEH phosphatase knock-in (KI) animals (rats). They unexpectedly revealed that inhibition of the phosphatase domain of sEH improves cardiac systolic function, decreases body weight and increases insulin sensitivity. Moreover under high fat diet, the animals have a decreased body weight gain, were protected against the development of insulin resistance, hepatic steatosis and cardiac hypertrophy. Inhibition of the phosphatase domain of sEH thus represents a new pharmacological target in the treatment of cardiometabolic diseases.

### FIELD OF THE INVENTION

The present invention relates to use of inhibitors of phosphatase activity of soluble epoxide for the treatment of cardiometabolic diseases.

### BACKGROUND OF THE INVENTION

The growing prevalence of obesity and type 2 diabetes complicates risk and clinical management by potentiating and/or exacerbating hypertension, hyperlipidemia, atherosclerosis and cardiomyopathy, leading to increasing use of the term “cardiometabolic disease” (CMD) to encompass the many facets of this complex syndrome. While several classes of drugs have been developed to manage various aspects of CMD, novel integrative therapies that target central “unifying” features of its pathogenesis and/or progression are needed to simplify clinical management, reduce risk of multi-drug interactions, and avoid potentially adverse effects.

Soluble epoxide hydrolase (sEH) is an ubiquitous bifunctional enzyme notably expressed in cardiovascular and metabolic tissues. The hydrolase domain of sEH metabolizes cardiovascular protective epoxyfatty acids involved in the regulation of endothelial function and in the maintain of cardiovascular homeostasis. Aside from its hydrolase activity, sEH also possesses a phosphatase domain whose function has been poorly investigated. While inhibition of the sEH hydrolase (sEH-H) activity has been widely investigated including in cardiovascular diseases, the role of the sEH phosphatase (sEH-P) activity has been poorly studied. Thus, inhibition of sEH-P represents a new therapeutic target to treat cardiometabolic diseases.

### SUMMARY OF THE INVENTION

The present invention relates to the use of inhibitors of phosphatase activity of soluble epoxide for the treatment of cardiometabolic diseases. In particular, the present invention is defined by the claims.

### DETAILED DESCRIPTION OF THE INVENTION

The inventors assessed the role of the sEH phosphatase domain in metabolism and cardiovascular system. They unexpectedly revealed that inhibition of the phosphatase domain of sEH could represent a new pharmacological target in the treatment of cardiometabolic diseases.

Accordingly, the first object of the present invention relates to a method of treating a cardiometabolic disease in a subject in need thereof comprising administering to the subject a therapeutically effective amount of an inhibitor of phosphatase activity of soluble epoxide hydro lase.

As used herein, the term “cardiometabolic disease” refers to a group of diseases, concerning the cardiovascular system and the metabolic system.

Examples of cardiometabolic diseases or disorders include, but are not limited to obesity, type 2 diabetes, metabolic syndrome, insulin resistance and dyslipidemias.

As used herein the term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

As used herein, the term “type 2 diabetes” or “non-insulin dependent diabetes mellitus (NIDDM)” has its general meaning in the art. Type 2 diabetes often occurs when levels of insulin are normal or even elevated and appears to result from the inability of tissues to respond appropriately to insulin. Most of the Type 2 diabetics are obese.

As used herein, the term “Metabolic Syndrome” refers to a subject characterized by having three or more of the following symptoms: abdominal obesity, hyperglyceridemia, low HDL cholesterol, high blood pressure, and high fasting plasma glucose. The criteria for these symptoms are defined in the third Report of the National Cholesterol Education Program Expert Panel in Detection, Evaluation and Treatment of High blood Cholesterol in Adults (Ford, E S. et al. 2002).

As used herein, the term “insulin resistance” has its common meaning in the art. Insulin resistance is a physiological condition where the natural hormone insulin becomes less effective at lowering blood sugars. The resulting increase in blood glucose may raise levels outside the normal range and cause adverse health effects such as metabolic syndrome, dyslipidemia and subsequently type 2 diabetes mellitus. The method of the present invention is thus particularly suitable for the treatment of type 2 diabetes. As used herein, the term “type 2 diabetes” or “non-insulin dependent diabetes mellitus (NIDDM)” has its general meaning in the art. Type 2 diabetes often occurs when levels of insulin are normal or even elevated and appears to result from the inability of tissues to respond appropriately to insulin. Most of the type 2 diabetics are obese.

As used herein, the term “dyslipidemia” refers to a disorder of lipid and/or lipoprotein metabolism, including lipid and/or lipoprotein overproduction or deficiency. Dyslipidemias may be manifested by elevation of lipids such as cholesterol and triglycerides as well as lipoproteins such as low-density lipoprotein (LDL) cholesterol. In particular, the term encompasses hypercholesterolemia, hyperlipidemia, and hypertriglyceridemia. As used herein the term “hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. (1988) 148, 36-39). As used herein, the term “hyperlipidemia” or “hyperlipemia” is a condition characterized by elevated serum lipids or circulating (plasma) lipids. This condition manifests an abnormally high concentration of fats. The lipid fractions in the circulating blood are cholesterol, low density lipoproteins, very low density lipoproteins and triglycerides. As used herein, the term “hypertriglyceridemia” means a condition characterized by elevated triglyceride levels.

The term “cardiometabolic disease” also encompasses cardiovascular complications, hepatic complications, respiratory complications, renal complications, nervous system complications and inflammation complications of obesity, type 2 diabetes, metabolic syndrome, insulin resistance and/or dyslipidemias.

Cardiovascular complications include cardiovascular disease (CVD), coronary artery disease, coronary heart disease (CHD), atherosclerosis, in particular iliac or femoral atherosclerosis, microangiopathy, angina pectoris, thrombosis, heart failure, stroke, vascular aneurysm, acute coronary syndromes such as myocardial infarction, vascular stenosis and infarction, vascular dementia and brain ischemia. Cardiovascular complications of diabetes include hypertension, cardiovascular disease (CVD) and brain ischemia. More particularly, cardiac complications include atherosclerosis, coronary heart disease, obesity-associated heart disease, insulin resistance-associated heart disease, hypertensive heart disease, cardiac remodelling, and heart failure.

Hepatic complications include in particular non-alcoholic fatty liver disease. As used herein, the term “non-alcoholic fatty liver disease” or “NAFLD” has its general meaning in the art and refers to one cause of a fatty liver, occurring when fat is deposited in the liver not due to excessive alcohol use. NAFLD is defined as the accumulation of fat in the liver, but not as secondary consequence of alcohol consumption. NAFLD can be sub-classified as non-alcoholic steatohepatitis (NASH) and nonalcoholic fatty liver (NAFL). Nonalcoholic fatty liver (NAFL) is a type of NAFLD and is a condition in which fat accumulates in the liver cells. NAFL has minimal risk of progressing to cirrhosis. Nonalcoholic steatohepatitis (NASH) is the more extreme form of NAFLD, and is regarded as a major cause of fibrosis and cirrhosis of the liver of unknown cause. The major feature in NASH is fat in the liver, along with inflammation and damage. NASH can be severe and can lead to fibrosis and cirrhosis, in which the liver is permanently damaged and scarred and no longer able to work properly.

As used herein, the term “treatment” or “treat” refer to both prophylactic or preventive treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]). In particular, the method of the present invention is particularly suitable for improving blood glucose control, enhancing insulin signalling in skeletal muscle and adipose tissue, reducing lipotoxicity in skeletal muscle and adipose tissue, increasing lipid oxidative capacity in skeletal muscle and adipose tissue, or maintaining long-term insulin sensitivity in the subject.

As used herein, the term “soluble epoxide hydrolase” or “sEH” has its general meaning in the art and refers to the ubiquitous enzyme encodes by the EPXH2 gene. The cloning, sequence, and accession numbers of the human sEH sequence are set forth in Beetham et al, Arch. Biochem. Biophys. 305(1):197-201 (1993). The amino acid sequence of human sEH is disclosed in U.S. Pat. No. 5,445,956.

As used herein, the term “inhibitor” as used herein includes drugs for inhibiting activity of the target molecule. The mode of the inhibitor is not particularly limited, and examples include e.g. low-molecular compounds, antibodies and aptamer. As used herein, the expression “inhibitor of phosphatase activity of soluble epoxide hydrolase” refers to any dug capable of inhibiting the phosphatase activity of the enzyme without inhibiting its hydrolase activity. Typically, the inhibitors are ligands for the amino terminus active site associated with the phosphatase activity of the enzyme. In some embodiments, the inhibitor of the present invention is a competitive inhibitor which allosterically alter the phosphatase activity of the enzyme. In some embodiments, the inhibitor of the present invention is non-competitive inhibitor which alter the phosphatase activity of the enzyme by modifying its configuration Assays for determining whether a compound would inhibit phosphatase activity of soluble epoxide hydrolase are well known in the art and typically include the methods disclosed in:
Enayetallah A E, Grant D F. Effects of human soluble epoxide hydrolase polymorphisms on isoprenoid phosphate hydro lysis. BiochemBiophys Res Commun. 2006 Mar. 3; 341(1):254-60.
Cronin A, Homburg S, Dürk H, Richter I, Adamska M, Frère F, Arand M. Insights into the catalytic mechanism of human sEH phosphatase by site-directed mutagenesis and LC-MS/MS analysis. J Mol Biol. 2008 Nov. 14; 383(3):627-40.
Hahn S, Achenbach J, Buscató E, Klingler F M, Schroeder M, Meirer K, Hieke M, Heering J, Barbosa-Sicard E, Loehr F, Fleming I, Doetsch V, Schubert-Zsilavecz M, Steinhilber D, Proschak E. Complementary screening techniques yielded fragments that inhibit the phosphatase activity of soluble epoxide hydrolase. ChemMedChem. 2011 Dec. 9; 6(12):2146-9.
Morisseau C, Sandeo S, Cortopassi G, Hammock B D. Development of an HTS assay for EPHX2 phosphatase activity and screening of nontargeted libraries. Anal Biochem. 2013 Mar. 1; 434(1):105-11. doi: 10.1016/j.ab.2012.11.017.
Klingler F M, Wolf M, Wittmann S, Gribbon P, Proschak E. Bacterial Expression and HTS Assessment of Soluble Epoxide Hydrolase Phosphatase. J Biomol Screen. 2016 August; 21(7):689-94.

In some embodiment, the inhibitor of phosphatase activity of soluble epoxide hydrolase is 4-(4-(3,4-Dichlorophenyl)-5-phenyloxazol-2-yl)butanoic acid and is having the following structure in the art:

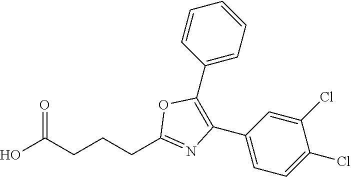 [SMILES: O=C(O)CCCc1nc(-c2ccc(Cl)c(Cl)c2)c(-c2ccccc2)o1]

In some embodiment, the inhibitor of phosphatase activity of soluble epoxide hydrolase is 3-(4-(3,4-Dichlorophenyl)-5-phenyloxazol-2-yl)benzoic acid and is having the following structure in the art:

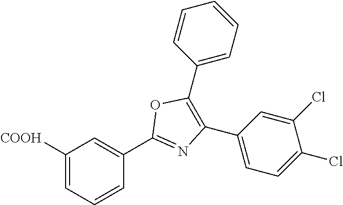 [SMILES: O=C(O)c1cccc(-c2nc(-c3ccc(Cl)c(Cl)c3)c(-c3ccccc3)o2)c1]

In some embodiment, the inhibitor of phosphatase activity of soluble epoxide hydrolase is sodium dodecyl sulfate and is having the following structure in the art:

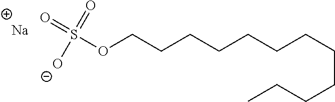 [SMILES: [Na+] ; CCCCCCCCCCCCOS(=O)(=O)[O-]]

In some embodiment, the inhibitor of phosphatase activity of soluble epoxide hydrolase is N-acetyl-S-farnesyl-L-cysteine and is having the following structure in the art:

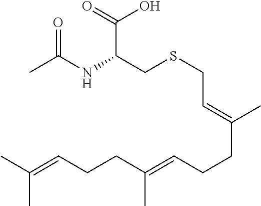 [SMILES: CC(=O)N[C@@H](CSC/C=C(\C)CC/C=C(\C)CCC=C(C)C)C(=O)O]

In some embodiment, the inhibitor of phosphatase activity of soluble epoxide hydrolase is SMTP-7 and is having the following structure in the art:

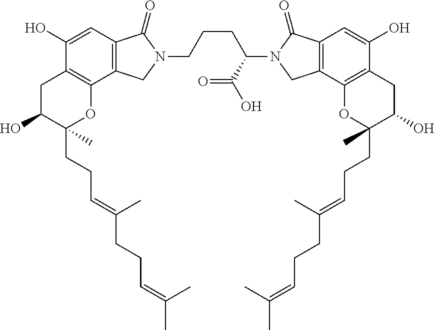 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

In some embodiment, the inhibitor of phosphatase activity of soluble epoxide hydrolase is ebselen and is having the following structure in the art:

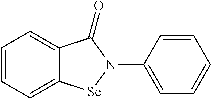 [SMILES: O=c1c2ccccc2[se]n1-c1ccccc1]

In some embodiment, the inhibitor of phosphatase activity of soluble epoxide hydrolase is oxaprozin and is having the following structure in the art:

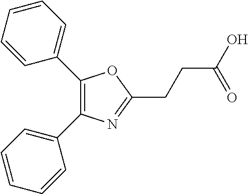 [SMILES: O=C(O)CCc1nc(-c2ccccc2)c(-c2ccccc2)o1]

In some embodiment, the inhibitor of phosphatase activity of soluble epoxide hydrolase is Fmoc-L-Phe(4-CN) and is having the following structure in the art:

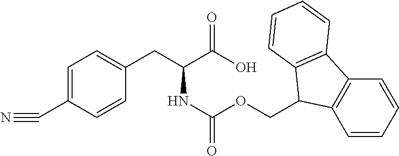 [SMILES: N#Cc1ccc(C[C@H](NC(=O)OCC2c3ccccc3-c3ccccc32)C(=O)O)cc1]

In some embodiment, the inhibitor of phosphatase activity of soluble epoxide hydrolase is Boc-L-Tyr(Bzl) and is having the following structure in the art:

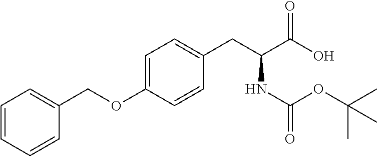 [SMILES: CC(C)(C)OC(=O)N[C@@H](Cc1ccc(OCc2ccccc2)cc1)C(=O)O]

In some embodiment, the inhibitor of phosphatase activity of soluble epoxide hydrolase includes the compounds disclosed in the publication: Kramer J S et al. (2019).

In some embodiment, the inhibitor of phosphatase activity of soluble epoxide hydrolase includes the compounds disclosed in the publication Matsumoto et al. (2019).

Exemplary inhibitors of phosphatase activity of sEH includes the compounds disclosed in the International Patent Application WO2007022059. In some embodiments, the inhibitor of the present invention is selected from the group consisting of compounds with the structure set forth below:

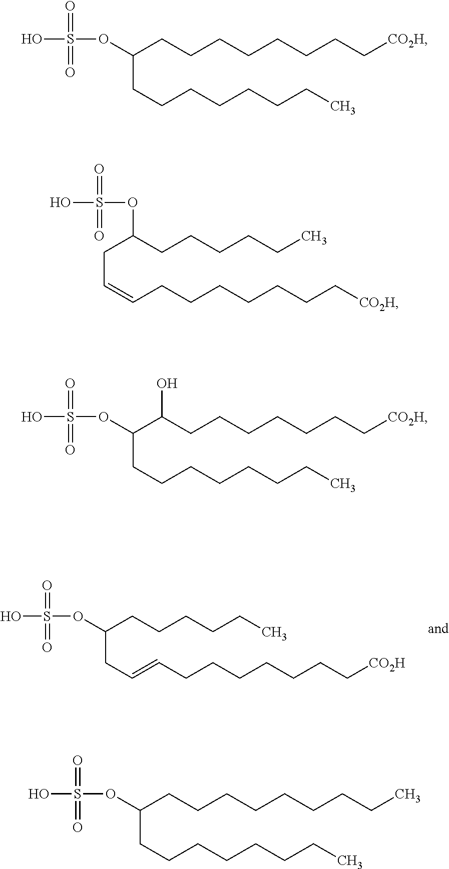 [SMILES: CCCCCCCCC(CCCCCCCCC(=O)O)OS(=O)(=O)O ; CCCCCCC(C/C=C\CCCCCCCC(=O)O)OS(=O)(=O)O ; CCCCCCCCC(OS(=O)(=O)O)C(O)CCCCCCCC(=O)O ; CCCCCCC(C/C=C/CCCCCCCC(=O)O)OS(=O)(=O)O ; CCCCCCCCCC(CCCCCCCC)OS(=O)(=O)O]

In some embodiments, the inhibitor of the present invention is a synthetic single domain antibody.

As used herein the term “single domain antibody” (hs2dAb) has its general meaning in the art and refers to the single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus yet another consequence of small size is that a nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in nanobodies being extremely thermostable, stable to extreme pH and to proteolytic digestion, and poorly antigenic. Another consequence is that nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDRl”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system amino acid numbering (http://imgt.cines.fr/).

As used herein, the term “synthetic” means that such antibody has not been obtained from fragments of naturally occurring antibodies but produced from recombinant nucleic acids comprising artificial coding sequences.

As used herein, the term “scaffold” refers to the 4 framework regions of the synthetic single domain antibody. Typically, all single domain antibodies have the same scaffold amino acid sequences while their CDRs may be different (the diversity of each library is only in the CDR regions).

As used herein, the term “amino acid sequence” has its general meaning and is a sequence of amino acids that confers to a protein its primary structure. According to the invention, the amino acid sequence may be modified with one, two or three conservative amino acid substitutions, without appreciable loss of interactive binding capacity. By “conservative amino acid substitution”, it is meant that an amino acid can be replaced with another amino acid having a similar side chain. Families of amino acid having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., glycine, cysteine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In one specific embodiment, the synthetic single domain antibody (hs2dAb) derives from VHH of Lama species and comprises the following humanized amino acid residues: F12, P15, S49, S81, K82, V85, Y86, S91, R93 and A94. In one related specific embodiment, the synthetic single domain antibody may comprise all the following amino acid residues: Q8, A9, F12, P15, F37, K43, E44, R45, F47, S49, A50, S81, K82, V85, Y86, S91, R93, A94, T99, Q108.

In one specific embodiment, the synthetic single domain antibody scaffold is obtained by mutagenizing a coding sequence of VHH of Lama species scaffold antibody in order to obtain at least the following humanized amino acid residues in the amino acid sequence: P15, S49, 10 S81, R93, A94, and preferably, the following humanized amino acid residues: F12, P15, S49, S81, K82, V85, Y86, S91, R93 and A94.

In one specific embodiment, the isolated single domain antibody comprising an amino acid sequence of formula (I) consisting in three complementary determining regions (CDR1 to CDR3) and four framework regions (FR1 to FR4): FR1-CDR1-FR2-CDR2-FR3-CDR3-FR4 (I).

| SEQ | | |
|---|---|---|
| ID | | |
| NO: | Description | Sequence |
| 1 | single domain | VQLQASGGGFVQPGGSLRLSCAASG |
| | antibody FR1 | |
| 2 | single domain | MGWFRQAPGKEREFVSAIS |
| | antibody FR2 | |
| 3 | single domain | YYADSVKGRFTISRDNSKNTVYLQMNSLRA |
| | antibody FR3 | EDTATYYCA |
| 4 | single domain | YWGQGTQVTVSS |
| | antibody FR4 | |

In one preferred embodiment the synthetic single domain antibody comprises the following framework regions consisting of FR1 of SEQ ID NO:1, FR2 of SEQ ID NO:2, FR3 of SEQ ID NO: 3 and FR4 of SEQ ID NO:4, or functional variant framework regions, for example with no more than 1, 2 or 3 conservative amino acid substitutions within each framework region, more preferably, within only one framework region.

In one preferred embodiment, the amino acids residues of the synthetic CDR1 and CDR2 are determined by the following rules:

at CDR1 position 1: Y, R, S, T, F, G, A, or D;

at CDR1 position 2: Y, S, T, F, G, T, or T;

at CDR1 position 3: Y, S, F, or W;

at CDR1 position 4: Y, R, S, T, F, G, A, W, D, E, K or N;

at CDR1 position 5: S, T, F, G, A, W, D, E, N, I, H, R, Q, or L;

at CDR1 position 6: S, T, Y, D, or E;

at CDR1 position 7: S, T, G, A, D, E, N, I, or V;

at CDR2 position 1: R, S, F, G, A, W, D, E, or Y;

at CDR2 position 2: S, T, F, G, A, W, D, E, N, H, R, Q, L or Y;

at CDR2 position 3: S, T, F, G, A, W, D, E, N, H, Q, P;

at CDR2 position 4: G, S, T, N, or D;

at CDR2 position 5: S, T, F, G, A, Y, D, E, N, I, H, R, Q, L, P, V, W, K or M;

at CDR2 position 6: S, T, F, G, A, Y, D, E, N, I, H, R, Q, L, P, V, W, or K;

at CDR2 position 7: S, T, F, G, A, Y, D, E, N, I, H, R, Q, L, P, or V;

In one related embodiment that may be combined with the preceding embodiment, said CDR3 amino acid sequence comprises between 9 and 18 amino acids. In one related embodiment that may be combined with the preceding embodiment, said CDR3 amino acid sequence comprises amino acid residues selected among one or more of the following amino acids: S, T, F, G, A, Y, D, E, N, I, H, R, Q, L, P, V, W, K, M.

In another embodiment, the synthetic single domain antibody scaffold comprises functional variants of FR1, FR2, FR3 and FR4 framework regions having at least 90%, preferably 95% identity to SEQ ID NOs 1-4 respectively.

According to the meaning of the present invention, the “identity” is calculated by comparing two aligned sequences in a comparison window. The sequence alignment allows determining the number of positions (nucleotides or amino acids) in common for the two sequences in the comparison window. The number of positions in common is therefore divided by the total number of positions in the comparison window and multiplied by 100 to obtain the identity percentage. The determination of the identity percentage of sequence can be made manually or thanks to well-known computer programs.

As used herein, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Myers and W. Miller (Comput. Appl. Biosci. 4: 1 1-17, 1988) which has been incorporated into the ALIGN program. In addition, the percent identity between two amino acid sequences can be determined using the Needleman and Wunsch (J. Mol. Biol. 48:443-453, 1970) algorithm which has been incorporated into the GAP program in the GCG software package. Yet another program to determine percent identity is CLUSTAL (M. Larkin ef a/., Bioinformatics 23:2947-2948, 2007; first described by D. Higgins and P. Sharp, Gene 73:237-244, 1988) which is available as stand-alone program or via web servers (see http://www.clustal.org/).

As used herein, the terms “purified” and “isolated” relate to the sdAb of the invention and mean that the sdAb is present in the substantial absence of other biologic macromolecules of the same type. The term “purified” as used here means preferably that at least 75% in weight, more preferably at least 85% in weight, even more preferably at least 95% in weight, and the more preferably at least 98% in weight of antibody, compared to the total weight of macromolecules present.

As used herein, the term “nucleic acid molecule” has its general meaning in the art and refers to a DNA or RNA molecule.

The method of making a synthetic single domain antibody library is as described in the patent application WO2015063331.

By a “therapeutically effective amount” is meant a sufficient amount of the inhibitor of the present invention for treating cardiometabolic disease at reasonable benefit/risk ratio. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the inhibitor of the present inventions; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250, 500 and 1,000 mg of the inhibitor of the present invention for the symptomatic adjustment of the dosage to the subject to be treated.

Typically the inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the inhibitor of the present inventions of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1. Compared to WT control rats, female and male sEH phosphatase knock-in (KI) rats have a decreased body weight (A), which is not related to modification in rat growth (B), and a reduction in body fat mass (C) and increased lean mass (D), taking into consideration their lower body weight (n=10-17 per group).

FIG. 2. Compared to WT control rats, sEH phosphatase knock-in (KI) rats have an increased glucose tolerance (A) and increased insulin sensitivity (B). Data from both female and male rats have been pooled. *P<0.05 vs. WT.

FIG. 3. Compared to WT male control rats, male sEH phosphatase knock-in (KI) rats have an increased basal body temperature measured by telemetry over a period of 48 h (A and B) and an increased heat production, representing thermogenesis, measured by temperature-sensitive transmitters implanted underneath the interscapular brown adipose tissue (BAT) during a cold test at 4° C. (C). *P<0.05 vs. WT.

FIG. 4. Male sEH phosphatase knock-in (KI) rats fed a high-fat diet (HFD; 60% fat) have a decreased body weight gain compared to male WT rats fed a HFD but also compared to male WT rats fed a normal food diet (NFD; A). In addition, KI rats fed a HFD were protected against the development of insulin resistance (B), hepatic steatosis (C) and cardiac hypertrophy (D) observed in WT rats fed a HFD (n=7-10 per group). *P<0.05 vs. WT NFD. ^{$}P<0.05 vs. WT HFD.

FIG. 5. Compared to WT control rats, sEH phosphatase knock-in (KI) rats have an elevation in left ventricular (LV) fractional shortening measured by echocardiography in anesthetized animals (A) as well as an increase LV developed pressure at baseline but during 5-min administrations of increasing doses of the beta-adrenergic agonist isoproterenol in isolated perfused heart (B). In addition, the cardiac necrosis area, representing infarct size, following ischemia-reperfusion was decreased in sEH phosphatase KI rats compared to WT rats (C). n=5-10 per group)

### EXAMPLE 1

Inhibition of sEH Phosphatase Induces a Lean Phenotype and Protects Against the Development of Diet-Induced Insulin Resistance and Related Complications

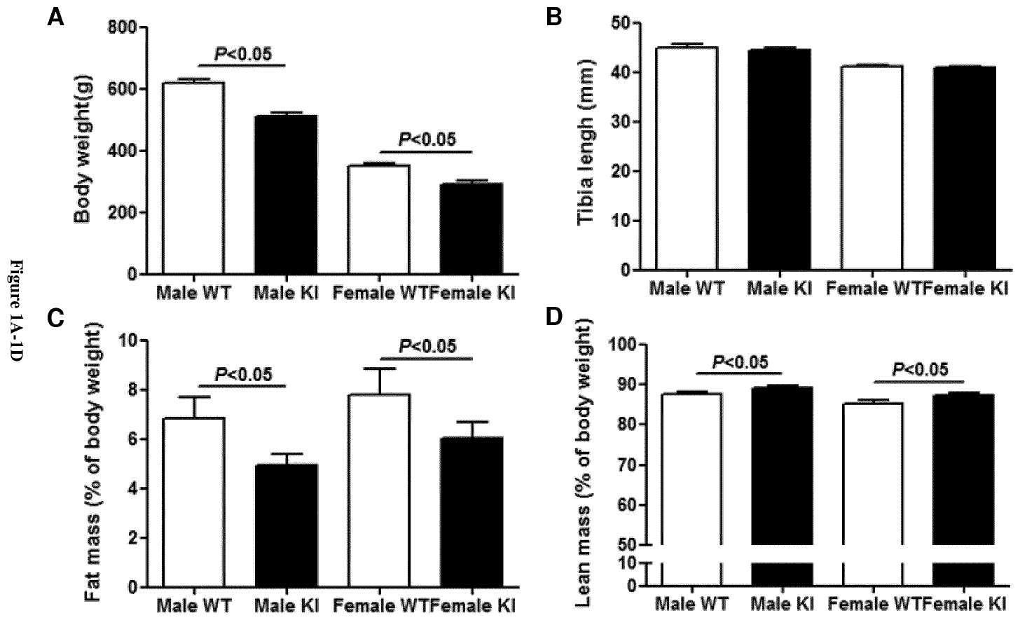

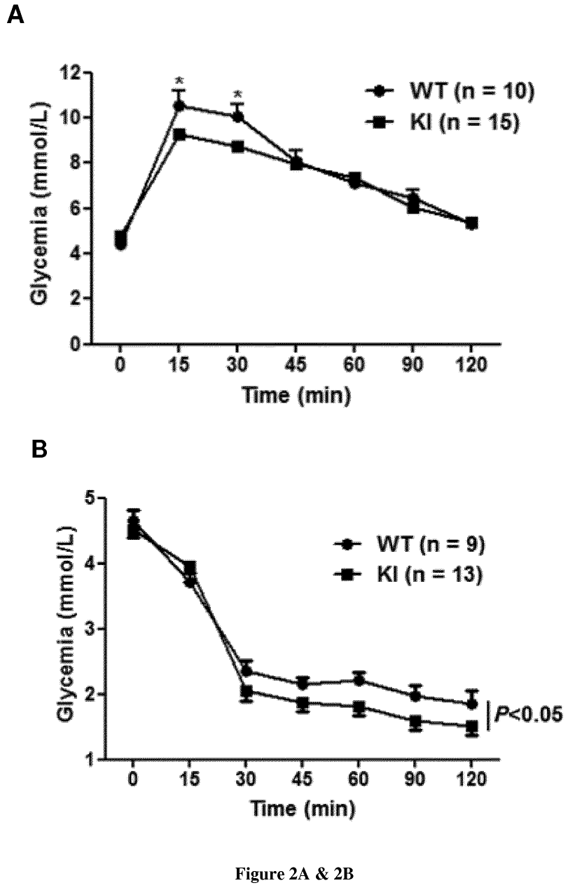

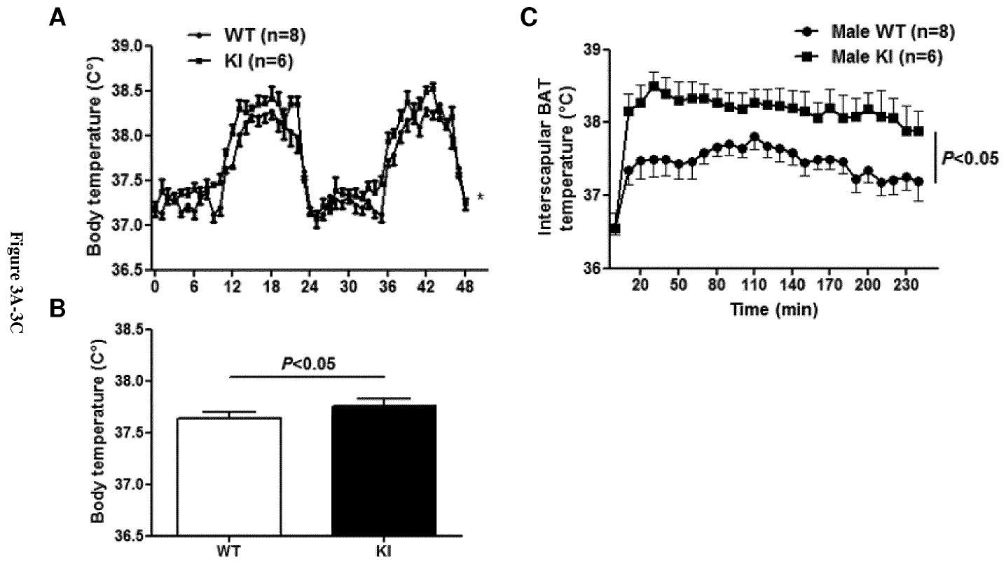

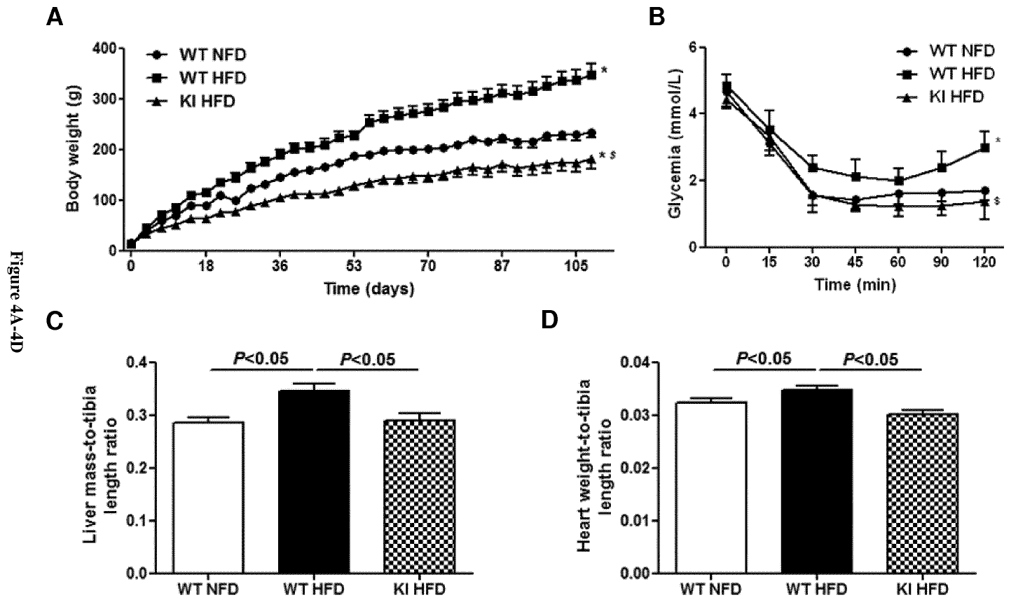

We generated sEH phosphatase knock-in (KI) rats. Compared to WT control rats the animals have a decreased body weight (FIG. 1A), which is not related to modification in rat growth (FIG. 1B), and a reduction in body fat mass (FIG. 1C) and increased lean mass (FIG. 1D), taking into consideration their lower body weight. Compared to WT control rats, sEH phosphatase KI rats have an increased glucose tolerance (FIG. 2A) and increased insulin sensitivity (FIG. 2B). Compared to WT male control rats, male sEH phosphatase KI rats have an increased basal body temperature measured by telemetry over a period of 48 h (FIGS. 3A and 3B) and an increased heat production, representing thermogenesis, measured by temperature-sensitive transmitters implanted underneath the interscapular brown adipose tissue (BAT) during a cold test at 4° C. (FIG. 3C). Finally, Male sEH phosphatase KI rats fed a high-fat diet (HFD; 60% fat) have a decreased body weight gain compared to male WT rats fed a HFD but also compared to male WT rats fed a normal food diet (NFD; FIG. 4A). In addition, sEH phosphatase KI rats fed a HFD were protected against the development of insulin resistance (FIG. 4B), hepatic steatosis (FIG. 4C) and cardiac hypertrophy (FIG. 4D) observed in WT rats fed a HFD.

### EXAMPLE 2

Inhibition of sEH Phosphatase Increases Cardiac Contractility and Protects the Heart Against Ischemia-Reperfusion Injury

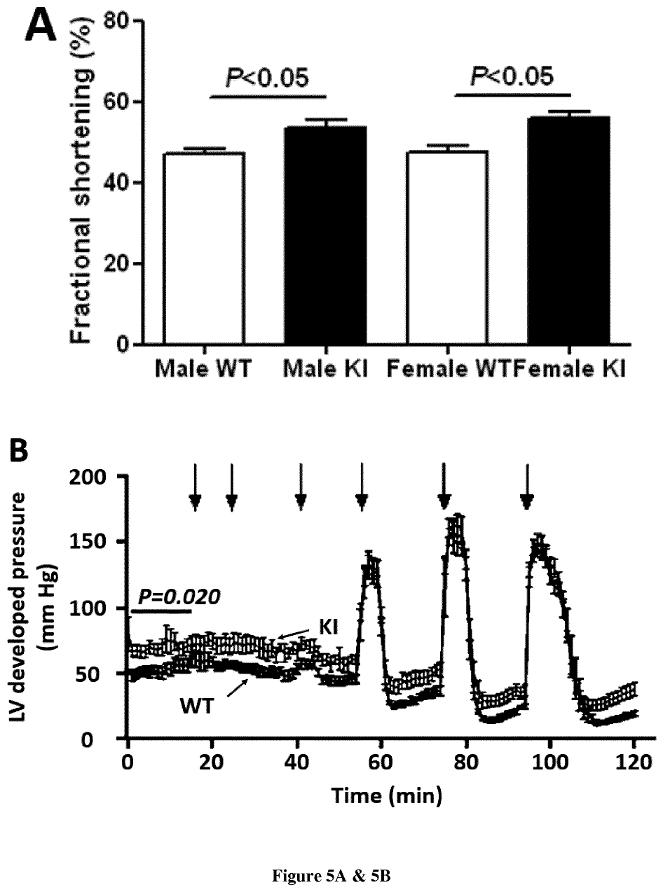

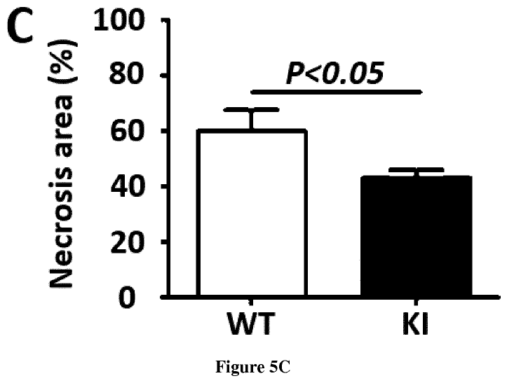

Telemetric monitoring showed no change in blood pressure but a trend for an increased heart rate during the active dark period in sEH-P KI rats compared to WT rats (Data not shown). Echocardiography showed that left ventricular (LV) posterior and anterior wall thicknesses were not significantly different between WT and sEH phosphatase KI rats but LV end-diastolic diameter but mostly LV end-systolic diameter were reduced in male and female KI rats compared to WT rats, leading to a significant increase in fractional shortening (FIG. 5A). Cardiac MRI confirmed that the reduction in LV volumes is associated to an increase in ejection fraction and actually lead to maintain stroke volume (Data not shown). Similar results were observed at the level of the right ventricle (Data not shown). The estimation of myocardial mass by cardiac MRI but mostly the weighting of the different heart cavities showed the decreased cardiac mass in sEH phosphatase KI rats (Data not shown). Moreover, the higher cardiac contractility was confirmed by the increase in LV developed pressure in isolated perfused heart from sEH KI rats at baseline but no during beta-adrenergic stimulation with isoproterenol (FIG. 5B). In addition, the cardiac necrosis area, representing infarct size, following ischemia-reperfusion was decreased in sEH phosphatase KI rats compared to WT rats (FIG. 5C). All these cardiac effects may be related to the potentiation of mitochondrial ATP production as shown by the increase in myocardial adenine nucleotide pool in LV tissues from sEH phosphatase KI rats (Data not shown).

All the results prompt us to consider that inhibition of the phosphatase activity of sEH would be suitable for the treatment of cardiometabolic diseases.

### REFERENCES

Throughout this application, various references describe the state of the art to which this invention pertains. The disclosures of these references are hereby incorporated by reference into the present disclosure.
Kramer J S, Woltersdorf S, Duflot T, Hiesinger K, Lillich F F, Knoll F, Wittmann S K, Klingler F M, Brunst S, Chaikuad A, Morisseau C, Hammock B D, Buccellati C, Sala A, Rovati G E, Leuillier M, Fraineau S, Rondeaux J, Hernandez Olmos V, Heering J, Merk D, Pogoryelov D, Steinhilber D, Knapp S, Bellien J, Proschak E. Discovery of first in vivo active inhibitors of soluble epoxide hydrolase (sEH) phosphatase domain. J Med Chem. (2019).
Covian R1, Balaban R S. Cardiac mitochondrial matrix and respiratory complex protein phosphorylation. Am J Physiol Heart Circ Physiol. (2018). H940-66.
Matsumoto N., Kataoka M., Hirosaki H, Morisseau C., D. Hammock B., Suzuki E., Hasumi K. N-Substituted amino acid inhibitors of the phosphatase domain of the soluble epoxide hydrolase. Biochemical and Biophysical Research Communications 515 (2019). 248-253

1. A method of treating a cardiometabolic disease in a subject in need thereof comprising administering to the subject a therapeutically effective amount of an inhibitor of phosphatase activity of soluble epoxide hydrolase.

2. The method of claim 1 wherein the cardiometabolic diseases is selected from the group consisting of obesity, type 2 diabetes, metabolic syndrome, insulin resistance and dyslipidemias.

3. The method of claim 1 wherein cardiometabolic diseases is selected from the group consisting of cardiovascular complications, hepatic complications, respiratory complications, renal complications, nervous system complications and inflammation complications of obesity, type 2 diabetes, metabolic syndrome, insulin resistance and/or dyslipidemias.

4. The method of claim 3 wherein cardiac complications include atherosclerosis, coronary heart disease, obesity-associated heart disease, insulin resistance-associated heart disease, hypertensive heart disease, cardiac remodelling, and heart failure.

5. The method of claim 3 wherein hepatic complications include nonalcoholic steatohepatitis.


SEQUENCE LISTING (4 sequences)

SEQ ID NO: 1 - protein, 25 aa, Artificial Sequence
  Synthetic single domain antibody FR1
VQLQASGGGFVQPGGSLRLSCAASG

SEQ ID NO: 2 - protein, 19 aa, Artificial Sequence
  Synthetic single domain antibody FR2
MGWFRQAPGKEREFVSAIS

SEQ ID NO: 3 - protein, 39 aa, Artificial Sequence
  Synthetic single domain antibody FR3
YYADSVKGRFTISRDNSKNTVYLQMNSLRAEDTATYYCA

SEQ ID NO: 4 - protein, 12 aa, Artificial Sequence
  Synthetic single domain antibody FR4
YWGQGTQVTVSS